(19) United States
(12) Patent Application Publication
(10) Pub. No.: US 2025/0346953 A1
(43) Pub. Date: Nov. 13, 2025

(54) CELL-FREE DNA CONCENTRATION IN HYPOTHERMIC MACHINE PERFUSATE AS A RAPID MARKER FOR KIDNEY GRAFT QUALITY

(71) Applicant: UNIVERSITY OF FLORIDA RESEARCH FOUNDATION, INCORPORATED, Gainesville, FL (US)

(72) Inventors: Ali ZARRINPAR, Gainesville, FL (US); Sergio DUARTE, Gainesville, FL (US); Narendra BATTULA, Gainesville, FL (US); Matthew F. WILLMAN, Gainesville, FL (US)

(21) Appl. No.: 18/870,030

(22) PCT Filed: Jun. 2, 2023
(86) PCT No.: PCT/US2023/024307

§ 371 Date: Nov. 27, 2024

Related U.S. Application Data
(60) Provisional application No. 63/348,102, filed on Jun. 2, 2022.

(51) Int. Cl. C12Q 1/6876; A01N 1/122; A01N 1/143; A01N 1/144; A01N 1/162; A01N 1/165; C12Q 1/6806; C12Q 1/6851

(52) U.S. Cl. CPC C12Q 1/6876; A01N 1/122; A01N 1/143; A01N 1/144; A01N 1/162; A01N 1/165; C12Q 1/6851; C12Q 1/6806; C12Q 2600/118

(57) ABSTRACT

A system for the measurement of cell-free DNA that comes off of a kidney pump (perfusate) prior to transplantation to evaluate the viability of the organ (quantification of donor injury) and the risk for primary graft dysfunction after transplantation. This use of cfDNA does not require sequencing just quantification. A device that would connect to a kidney pump and directly measure the cfDNA and analyze it.

### BACKGROUND

Advances in allograft preservation within the last century have transformed the field of solid organ transplantation. For example, in renal transplantation, the widespread use of hypothermic machine perfusion (HMP) has become standard of care in kidney preservation, maintaining organ viability in the transition from donor to recipient and greatly reducing postoperative delayed graft function in the recipient compared to static cold storage (SCS).^{1,2,3} Additionally, HMP allows for longer storage times compared to SCS,⁴ providing the opportunity to continuously assess intrinsic graft attributes and levels of tissue damage that contribute to poor graft function, which is associated with recipient morbidity and mortality.⁵ Allografts continue to accrue injury during the preservation period, and as such the quality of the graft upon procurement will not be the same after 24 to 48 hours of preservation. Thus, demand is high for methods to assess graft quality repeatedly during the preservation period and to predict graft function after transplantation.

In renal allografts preserved with HMP, methods of predicting donor organ function have had marginal success in reliably predicting organ damage and subsequent graft function. Currently, the most widely used method relies on dynamic parameters measured by the perfusion machine, namely, vascular resistance and flow, where flow correlates positively with graft function and resistance correlates negatively.⁶ However, the use of these indices to guide acceptance or rejection of donated kidneys has been criticized, as many allografts that may have been discarded on the basis of unacceptable pump parameters later demonstrate sufficient function after transplantation.^{7,8,9}

To aid decision-making regarding donor organ acceptance, new avenues are being explored. Some have adopted a composite score assessing macroscopic appearance, flow, and urine output, when kidney grafts are subject to normothermic perfusion.¹⁰ Additional studies have focused on measuring biomarkers in the HMP perfusate, such as lactate dehydrogenase, aspartate transaminase, glutathione-S-transferase, interleukin-18, and other surrogates for glucose metabolism, oxygen consumption, glycolytic activity, ATP depletion, and mitochondrial damage.^{11,12,13} However, none of these dynamic values or biomarkers have adequate power to predict post-transplant kidney function.^{14,15} To date, a reliable method of directly measuring renal allograft injury while undergoing HMP that can also predict postoperative renal function has not been established.

### BRIEF DESCRIPTION OF THE DRAWINGS

The present embodiments are illustrated by way of example, and not by way of limitation, in the figures of the accompanying drawings.

The following figures are illustrative only, and are not intended to be limiting FIG. 1. Study Methods. The perfusate of human kidneys undergoing hypothermic machine perfusion was sampled, and soluble DNA (sDNA) was extracted and quantified. After the kidney was transplanted into the recipient, post-transplant outcomes and measures of kidney function were assessed.

FIG. 2. Handoff sDNA vs measurements of early graft function. Handoff sDNA negatively correlated with creatinine reduction ratio (CRR) on all postoperative days (POD) (A-D). Handoff sDNA showed a similar negative correlation with estimated glomerular filtration rate (eGFR) on POD3 (E) and POD4 (not shown), as well as a positive correlation with POD3 (F) and POD4 Creatinine.

FIG. 3. Soluble DNA levels in predicting delayed graft function (DGF). Handoff (A) and 5 min (B) sDNA concentration was significantly higher in the five individuals who exhibited DGF. The receiver operating characteristic (ROC) curves for our cohort demonstrate that sDNA as a biomarker for DGF has a reliable prognostic performance with an area under curve (AUC) of 0.816 for samples obtained at handoff (C) and AUC=0.771 for samples obtained at 5 min perfusion (D). Conversely, the ROC curves for Kidney Donor Profile Index (KDPI, E), renal vascular flow (F), and renal vascular resistance (G) were less reliable and were not statistically significant (KDPI AUC=0.748, p=0.074; flow AUC=0.600, p=0.485; resistance AUC=0.633, p=0.330).

FIG. 4 depicts an embodiment of a system for ex vivo organ perfusion.

FIG. 5. Graphical representation of dynamic hypothermic machine perfusion (HMP) parameters vs sDNA levels in the perfusate. There was significant correlation between 5 min sDNA with renal flow at 2 hrs and at 4 hrs (A, B), and between handoff sDNA and renal flow at 2 hrs (C), 4 hrs (not shown), and at handoff (D).

FIG. 6. Five-minute sDNA concentration vs creatinine reduction ratio (CRR). 5 min sDNA negatively correlated with CRR on postoperative day (POD) 2 (A) and POD4 (B).

### DETAILED DESCRIPTION

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described. All publications mentioned herein are incorporated herein by reference.

Generally, nomenclatures used in connection with, and techniques of, cell and tissue culture, molecular biology, immunology, microbiology, genetics, protein, and nucleic acid chemistry and hybridization described herein are those well-known and commonly used in the art. The methods and techniques of the present invention are generally performed according to conventional methods well known in the art and as described in various general and more specific references that are cited and discussed through the present specification unless otherwise indicated.

The term “about” means plus or minus 1%, 2%, 3%, 4%, 5%, 6%, 7%, 8%, 9%, or 10% of the number to which reference is being made.

The term “organ perfusion” refers the maintenance of a transplantation graft in a metabolically active state outside the body. Machine perfusion is a method involving organ perfusion with a controlled flow of perfusate. It facilitates the maintenance of organ microvasculature tone, provision of oxygen and nutrients in support of tissue metabolism, and removal of toxic metabolic waste. Different temperatures have been investigated for ex vivo machine perfusion, including normothermic machine perfusion (NMP) at 35-38° C., subnormothermic machine perfusion (SNMP) at 20-34° C., controlled oxygenated rewarming (COR) at 8-20° C., and hypothermic machine perfusion (HMP) at 0-8° C. The term “organ perfusate” refers to the plasma or serum circulated through the organ to maintain stable function ex vivo. Examples of perfusates include, but are not limited to, UW solution, Bretschneider solution, BES-HMP solution, and BGP-35 solution.

As used herein, the term “cell-free DNA” or “cfDNA” refers to the cell-free (non-encapsulated) DNA derived from apoptosis or necrosis of allograft tissue, which circulates in the body fluids of patients after organ transplantation. This measurement is a proxy for the health of the donor tissue. cfDNA is also referred to herein and is interchangeable with “soluble DNA” or “sDNA”.

The terms “quantify” or “quantification” refer to determining the average concentration of a molecule in a sample. In a specific embodiment, the device or the method is quantifying cfDNA in organ perfusate. The methods for quantification of cfDNA are well known in the arts and include spectrophotometric and fluorometric techniques or digital PCR.

The term “control level” relative to cfDNA in a pretransplant tissue or organ refers to a level of cfDNA in perfusate of an organ or tissue within 5 minutes of initiating perfusion, or a level of cfDNA in perfusate as predetermined by measuring perfusate from a plurality of organs or tissues undergoing perfusion that that did not exhibit delayed graft function upon transplantation.

### Overview

The acceptance of donor kidneys is left to the discretion of the transplant physicians. This can be a daunting task, as the recipient's health and quality of life is significantly dictated by the outcome of this decision.²⁰ Several factors, such as KDPI, appearance, ischemia time, histologic features of any biopsy, and dynamic values such as RR and RF produced during HMP, are used to inform the surgeon's decision. However, there is no quantitative, noninvasive, repeatable biomarker of tissue damage correlating with postoperative renal function available to aid in the decision-making process. In this study, we propose sDNA measured in HMP perfusate of renal allografts as one such biomarker.

Soluble DNAs are cell-free circulating, short fragments of DNA released from injured, necrotic, apoptotic, and other dying cells. Soluble DNA concentration can be measured in plasma and is revolutionizing many medical fields such as oncology, maternal fetal medicine, and transplantation.^{21,22,23} The measurement of DNA has become a useful practice in determining allograft integrity in the post-transplant setting.²⁴ Several studies have validated that donor-derived cell-free DNA can be quantified in the bloodstream of renal transplant recipients and used as a surrogate for graft injury.^{25,26,27} As it has been shown to be a biomarker for graft damage in the recipient bloodstream, it is likely that measurement of soluble DNA in the perfusate during HMP could provide insight as to graft damage prior to transplantation. This was very recently demonstrated in pulmonary allografts,¹⁶ but has not been shown within human donor kidneys for which HMP is widely established as a clinical standard, until now.

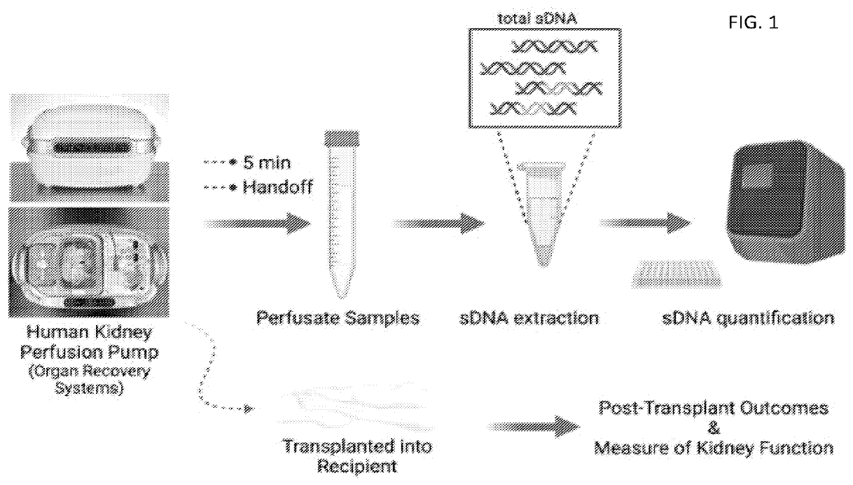

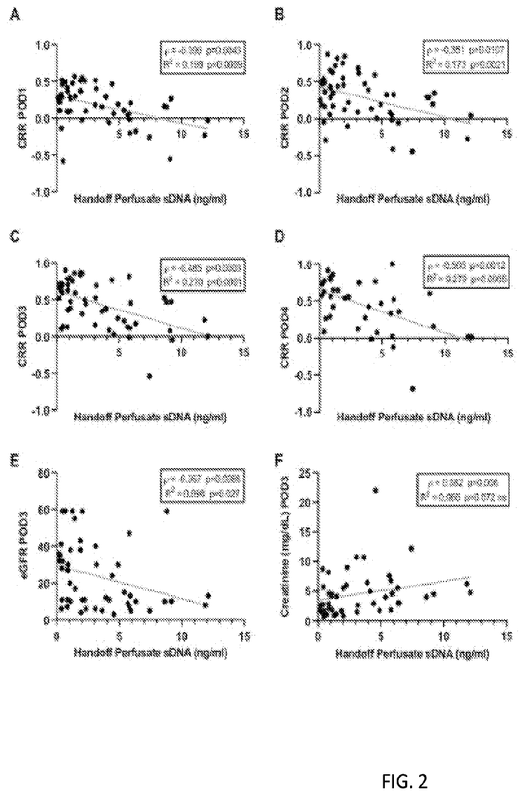

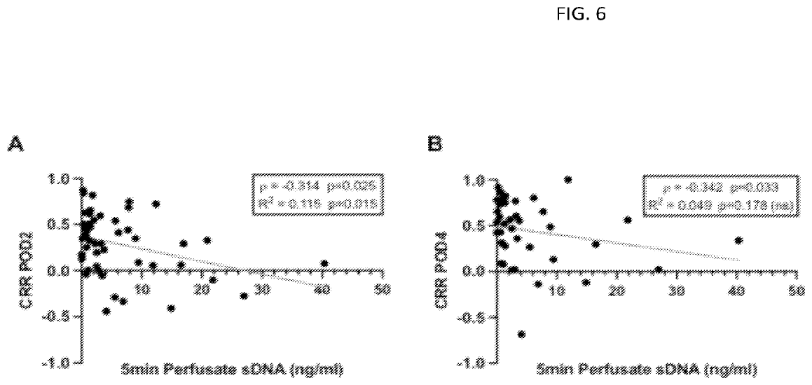

Here, it is confirmed that sDNA is a reliable measure of early graft function in a post-transplant population. The disclosed data demonstrates that sDNA within the HMP perfusate correlates with markers of renal function post-transplant. Specifically, higher 5-minute sDNA concentrations correspond with lower CRR on POD2 and POD4 (Table 6, FIG. 6), and a higher level of sDNA at handoff significantly correlates with a lower CRR on all postoperative days (Table 4, FIG. 2). The rate of creatinine clearance post transplantation is a more relevant assessment of short-term graft function than simple creatinine levels. CRR is an accurate of measure of this rate. Additionally, CRR calculated on POD2 has previously been shown to predict long-term graft outcomes, specifically serum Cr at one year and at 5 years post-transplant.^{28,29,30} In comparison, none of the commonly employed predictors of a graft's suitability (KDPI, RR, and RF) were significantly correlated with CRR on any postoperative day in this study (Table 3). It is noted that higher KDPI was significantly correlated with lower eGFR in the studies disclosed herein (Table 3), supporting its continued use to guide decisions regarding graft suitability in conjunction with other parameters such as sDNA concentration.

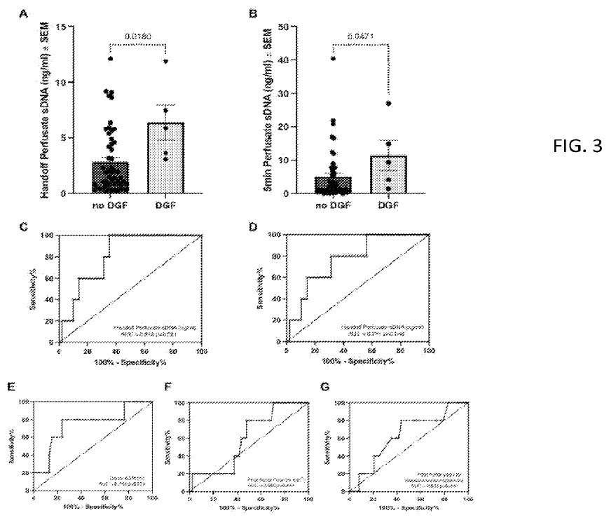

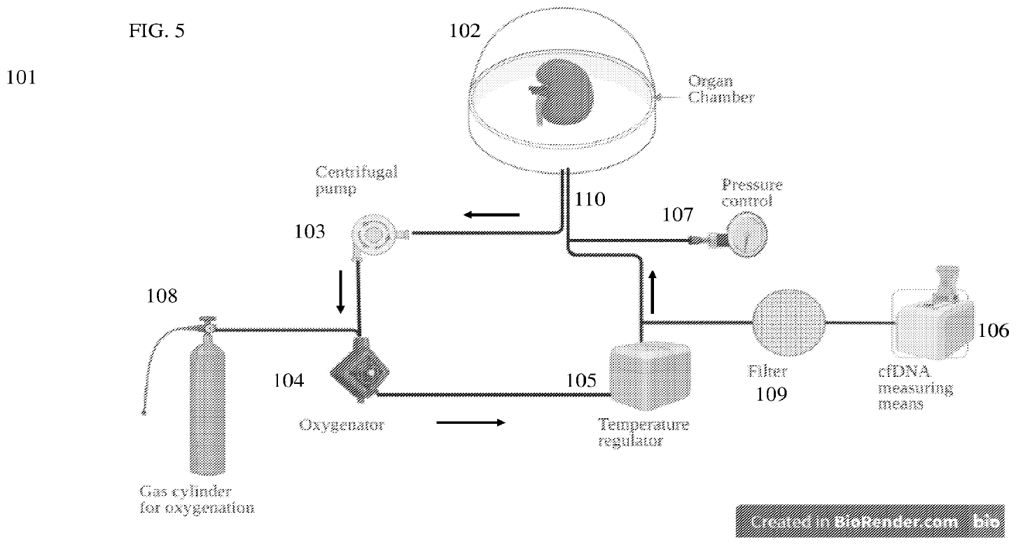

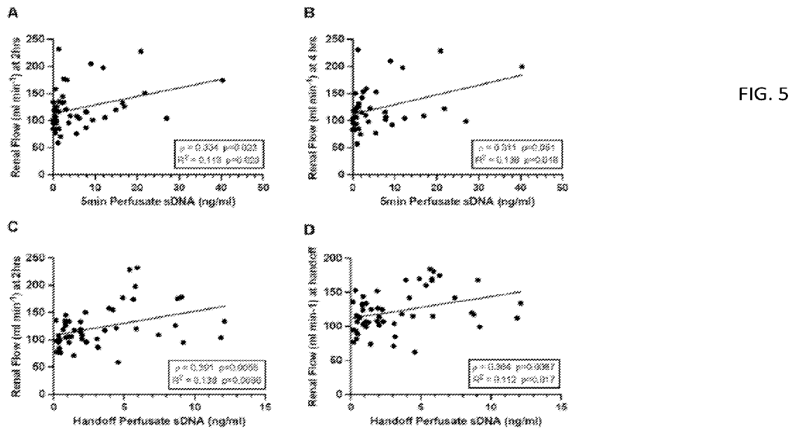

Curiously, it was observed that renal flow is positively correlated with sDNA concentration, while renal resistance is negatively correlated (Table 5, FIG. 5). While counterintuitive, it is surmised that higher flow during HMP allows for greater exposure of damaged tissue to the HMP perfusate, allowing the nuclear-origin DNA to solubilize within the solution and thus be detected at higher concentration. This relationship between sDNA and pump parameters may need further optimization, but the disclosed findings nonetheless indicate that higher sDNA within HMP perfusate is associated with worse post-transplant outcomes, particularly lower eGFR and CRR (Table 6, FIG. 6, Table 4, FIG. 2) as well as higher rates of DGF (FIG. 3), where RR and RF have previously proved unreliable.

Accordingly, the present disclosure is based on the discovery that the concentration of sDNA in the perfusate of ex vivo hypothermic perfused kidney grafts provides insight on the quality of these grafts at the time of transplantation. Furthermore, the sDNA levels are directly correlated with early post-transplant renal function.

In certain embodiments, provided is a system for organ perfusion that quantifies the cfDNA in the organ perfusate. Embodiments of the disclosure include a device for measuring cfDNA in the organ perfusate and a method for determining pre-transplant quality of the organ.

Presented in FIG. 4 is a diagram embodiment of a system 101 for organ perfusion that quantifies the cfDNA in the organ perfusates. The system comprises an organ chamber 102 for holding the organ and perfusate. The perfusate is moved through the system 101 by a pump 110 that connects all the components of the system. After the perfusate leaves the organ chamber 102, it passes through the pump means 103. The pump means 103 controls the flow rate of the perfusate through the pump. In certain embodiments, the flow rate comprises 20 ml/min to 150 ml/min.

The perfusate continues to the oxygenator 104 which provides oxygen, nitrogen, carbon dioxide, or any combination thereof to the perfusate. Optionally, the oxygenator 104 is connected to a gas source 108 or can be connect to a gas source.

Typically, perfusion conducted is hypothermic organ perfusion. In order to maintain a temperature range of 0° C. to 12° C., the perfusate flows through a temperature regulator 105. The perfusate continues back to the organ chamber and the pressure level is measured and regulated by a pressure controller 107. In certain embodiments, the pressure level of the perfusate is between 10 to 100 mmHg 20. In a more certain embodiment, the ORS pressure range is 10-65 mmHg.

The system contains a cfDNA detector 106 to determine the level of cfDNA in the perfusate. In certain embodiments, the perfusate is filtered before going to the cfDNA detector 106. The filter 109 removes possible contaminants from the perfusate that might interfere with the quantification of the cfDNA. Possible contaminants include, but are not limited to, proteins, phenol, or RNA.

### Organs and Tissues

Transplanted tissues and organs can be any allograft, including solid organs (such as kidney, liver, heart, lungs, pancreas, stomach, intestine, thymus, uterus, testis, ovaries, colon, spleen, parathyroid glands, and the like), tissues (such as bone marrow, bone, cornea, skin, heart valves, nerves, veins, tendons, pancreatic islets, blood, hand, face, skin, beta cells, parathyroid cells, limbs, and the like). Preferred transplants are kidney, heart, lung, intestines, liver, and pancreas. In an exemplary embodiment, the transplant tissue is a kidney.

cfDNA Detection

Disclosed is a device for detecting or determining the level of cfDNA in an organ perfusate. In certain embodiments, a cfDNA detector is connected to a perfusion machine or perfusion system. Optionally, the detector may contain a filter to remove contaminants that would interfere with quantifying the cfDNA.

In certain embodiments, the level of cfDNA can be determined in a perfusate using known techniques, from which the level of gene expression can be inferred. Levels of cfDNA can be quantitatively measured by Southern blotting which gives size and sequence information about the cfDNA molecules. A sample of cfDNA is separated on an agarose gel and hybridized to a radioactively labeled probe that is complementary to the target sequence. Or more typically real-time quantitative PCR (qPCR) is used. The cfDNA template is amplified in the quantitative step, during which the fluorescence emitted by labeled hybridization probes or intercalating dyes changes as the DNA amplification process progresses. With a carefully constructed standard curve, qPCR can produce an absolute measurement of the number of copies of original cfDNA, typically in units of copies per nanoliter of homogenized tissue or organ perfusate. qPCR is very sensitive.

In certain embodiments, the level of cfDNA can be determined in a perfusate using digital polymerase chain reaction (dPCR) implemented in the cfDNA detector. Digital PCR builds on traditional PCR amplification and fluorescent-probe-based detection methods to provide highly sensitive absolute quantification of nucleic acids without the need for standard curves. In a Droplet Digital™ PCR system, a PCR sample is partitioned into 20,000 droplets. After amplification, droplets containing cfDNA are detected by fluorescence and scored as positive, and droplets without fluorescence are scored as negative. Poisson statistical analysis of the numbers of positive and negative droplets yields absolute quantitation of the cfDNA.

In certain embodiments the method used to quantitate cfDNA in the organ perfusate is spectrophotometric analysis using a spectrophotometer implemented with the detector. A spectrophotometer determines the average concentrations of the nucleic acids DNA present in a mixture, as well as the purity. Spectrophotometric analysis of DNA is based on the principles that nucleic acids absorb ultraviolet light in a specific pattern. In the case of DNA, a sample is exposed to ultraviolet light at a wavelength of 260 nanometers (nm) and a photodetector measures the light that passes through the sample. Some of the ultraviolet light will pass through and some will be absorbed by the DNA. The great amount light absorbed by the sample, the higher the nucleic acid concentration in the sample. The resulting effect is that less light will strike the photodetector, and this will produce a higher optical density (OD). Using the Beer-Lambert law it is possible to relate the amount of light absorbed to the concentration of the absorbing molecule. At a wavelength of 260 nm, the average extinction coefficient for single-stranded DNA it is 0.027 (μg/ml)⁻¹ cm⁻¹. The spectrophotometer is calibrated with the perfusate solution before the organ perfusion begins.

An alternative method to assess cfDNA concentration is a fluorescent tag, which is a fluorescent dye used to measure the intensity of the dyes that bind to nucleic acids and selectively fluoresce when bound. This method is useful for cases where concentration is too low to accurately assess with spectrophotometry and in cases where contaminants absorbing at 260 nm make accurate quantitation by that method impossible.

### EXAMPLES

### Example 1. Perfusate Cell-Free DNA Content is a Potential Marker of Cellular Injury During

Hypothermic Machine Perfusion of Porcine Kidneys Subject to Prolonged Warm Ischemia Despite the potential benefits and expanding use of advanced dynamic perfusion systems in clinical practice, to date there is no robust and non-Invasive method to assess graft preservation quality. As it is released from injured, necrotic, and apoptotic cells, cell-free DNA (cfDNA) is a promising biomarker of tissue injury. This study was designed to test whether quantification of cfDNA concentration in the organ perfusate can serve as an accurate and efficient measure of tissue Injury and cell death in donation after circulatory death (DCD) kidney allografts undergoing ex-vivo hypothermic machine perfusion.

To mimic DCD kidney grafts, 8 porcine kidneys were subjected to 3 hours of warm ischemia, flushed with Plasmalyte, and perfused with non-oxygenated UW at 4° C. for 48 hours on a peristaltic pump. Tissue biopsies and perfusate samples were collected for biochemical and histological analysis and quantification of cfDNA of nuclear origin by qPCR after 1, 12, 24, and 48 hours.

CfDNA levels increased steadily in the perfusate over the course of hyperthermic perfusion, with the highest cfDNA content consistently observed after 48 h. The rise of cfDNA content was associated with a progressive rise in histological features of tissue necrosis throughout perfusion. Furthermore, increments in perfusate cfDNA levels were also associated with higher levels of tissue pro-apoptotic caspase-3 proteolytic activation and increased detection of terminal deoxynucleotidyl transferase dUTP nick end labeling (TUNEL) positive tubular and glomerular apoptosis.

The study provides evidence for the applicability of cfDNA monitoring of the perfusate of ex vivo hypothermic kidneys as an accurate and quantitative marker of tissue integrity and cellular injury in a preclinical pig model.

### Example 2. Cell-Free DNA Concentration in Hypothermic Machine Perfusate is a Potential Rapid Marker for Kidney Graft Quality

The successful use of hypothermic machine perfusion (HMP) as a clinical modality for graft preservation prior to kidney transplantation (KTx) has generated a demand for novel strategies aimed at improving graft viability assessment and facilitating prediction of early post-KTx graft function and survival. Circulating cell-free DNA (cfDNA) released from injured, necrotic, and apoptotic cells is an emerging biomarker of tissue injury. Here it was tested if cfDNA levels in the perfusate of human kidney allograft undergoing HMPIs an accurate measure of preservation quality and post-transplant renal function.

Perfusate samples of 12 kidney grafts selected for HMP at the University of Florida were collected after 5 minutes and at conclusion of HMP. Graft recipients were enrolled under an IRB approved protocol. cfDNA was quantified by real-time polymerase chain reaction using novel GAPDH primers (forward primer: TGGTGAAGCAGGCGTCG; reverse primer: GGTGTCGCTGTTGAAGTCAGA) for DNA of nuclear origin and correlated with both HMP parameters and post-kidney transplant clinical outcomes. The primary outcome was delayed graft function (DGF) and secondary outcomes were clinical measures of early graft function up to post-operative day (POD) 4.

Kidney grafts included in this assessment had a mean 597 min (SD: 368) of static preservation on ice, a mean 486 min (SD:315) of HMP and a mean 1086 min (SD:459) of total cold ischemia. There were no cases of DGF. 5 min perfusate cfDNA levels correlated positively with the graft's initial renal flow on the pump (p=0.623, p=0.0034) and negatively with the graft's Initial renal resistance on the pump (p=−0.592, p=0.0075). Interestingly, grafts with higher levels of perfusate cfDNA at HMP conclusion had reduced graft function in the initial post-KTx period. Increased endpoint perfusate cfDNA concentrations correlated with increased recipient serum creatinine levels at POD4 (R²=0.431, p=0.0057), decreased POD4 estimated glomerular filtration rate (R²=0.389, p=0.0098) and decreased creatinine reduction ratio (R²=0.674, p<0.0001). In contrast, there was no observed correlation between clinical measures of early post-KTX graft function and endpoint renal resistance or renal flow readings for these grafts

These preliminary findings provided initial evidence that quantification of cfDNA content in the perfusate of ex vivo hypothermic perfused kidney grafts can provide insight to the quality of preservation of these grafts and their early post-transplant renal function.

### Example 3: Soluble DNA Concentration in the Perfusate is a Predictor of Post-Transplant Renal Function in Hypothermic Perfused Kidney Allografts

### Methods

### Study Design

This is a single-center, prospective cohort study approved by the Institutional Review Board at the University of Florida (IRB #202001674). All human kidneys preserved by HMP as standard-of-care between July 2021 and December 2022 were included. Kidneys intended for pediatric (<18 years) recipients, discarded kidneys that were not transplanted, kidneys that were pumped at another institution prior to arriving at our institution, and kidneys involved in multi-organ transplants were excluded. Fifty-two kidneys and recipients were included in this study. HMP parameters measured and registered for this study were renal vascular resistance (RR) and flow (RF) at initiation, 2 hours, 4 hours (if applicable), and endpoint of perfusion. The primary endpoint of the study was delayed graft function (DGF), defined as the need for dialysis within the first 7 days after transplantation. Secondary endpoints were post-transplant clinical outcomes indicative of early graft function such as estimated glomerular filtration rate (eGFR), creatinine (Cr), and creatinine reduction ratio (CRR, defined in Equation 1), which were measured on postoperative days (POD) 1, 2, 3, and 4. We restricted our analysis of early graft function to the first 4 postoperative days at the end of which most of the study subjects are discharged at our institution; the clinical outcomes datasets and study N decreases for each subsequent time point. A schematic of the study methodology is depicted in FIG. 1.

$$Creatinine Reduction Ratio CRR = \frac{Creatinine Day X - Creatinine Pre Transplant}{Creatinine Day X} \qquad Equation 1$$

### Clinical Ex Vivo Kidney Machine Perfusion Procedure

Kidneys utilized during the study period underwent HMP using the LifePort Kidney Transporter 1.1 (Organ Recovery Systems, Itasca, IL), according to manufacturer instructions and as described in previous investigations.^{18,19} UW Machine Perfusion solution was used as perfusate. Perfusate samples of the kidney grafts selected for HMP were collected after 5 minutes of perfusion and at the conclusion of HMP at graft handoff to the surgical team for implantation.

sDNA Extraction and PCR Quantification

The sDNA within each HMP perfusate sample was isolated using the QIAamp MinElute ccfDNA Mini Kit (QIAgen Group, Germantown, MD) according to manufacturer instructions.

Briefly, 2 mL perfusate was added to the proprietary magnetic bead suspension, which allows for binding of cell-free DNA to magnetic beads. The bound cell-free DNA was then eluted from the beads and purified using the QIAamp MinElute membrane. Purified cell-free DNA eluted from the membrane is the resulting soluble DNA (sDNA) sample. The nuclear-origin sDNA within the eluate was then quantified by real-time polymerase chain reaction (RT-PCR) using specific primer sequences for DNA of nuclear origin (customized oligonucleotide targeting GAPDH gene, Integrated DNA Technologies, Coralville, Iowa).

### Statistical Analysis

Statistical analysis was performed using the R software package (V.4.1.3, The R Foundation for Statistical Computing). Non-parametric Spearman correlations were used to assess the relationships (direction and strength) between sDNA concentration and HMP parameters, as well as between sDNA concentration and postoperative variables. Linear regression analysis was used to assess the effect of sDNA concentration on these outcomes.

### Results

### Graft, Donor and Recipient Characteristics

A total of 52 kidneys and 52 recipients were studied. Demographic characteristics of donors and recipients are reported in Table 1. The majority of donor allografts were obtained from male (60.8%) donors after brain death (75.5%). Donor kidneys had a mean 640±309 min of static preservation on ice prior to placement on HMP, a mean 477±256 min of HMP, and a mean 1127±405 min of total cold ischemia. There was a significant relationship only between perfusate sDNA concentration at HMP conclusion (hereafter referred to as handoff) and total cold ischemia time (Table 2; p=0.3049). There were five cases of DGF among the recipients within the study period.

TABLE 1
Donor and recipient demographics. Some donor kidney totals
(n) do not add up to the total number of transplants performed
(n = 52), as this information was absent from the kidney
donor information sheet at the time of data collection.
Recipients

| | |
|---|---|
| Age (years ± SD) | 53 ± 15 |
| Sex n (%) | |
| Male | 32 (61.5) |
| Female | 20 (38.5) |
| Ethnicity n (%) | |
| Non-Hispanic White | 25 (48.1) |
| Hispanic | 7 (13.5) |
| Non-Hispanic Black | 19 (36.5) |
| Pacific Islander | 1 (1.9) |
| BMI (kg/m² ± SD) | 28.26 ± 5.40 |
| Baseline Cr (mg/dL ± SD) | 8.66 ± 4.69 |
| Preperative eGFR (mL/min ± SD) | 9.94 ± 10.16 |

Donors

| | |
|---|---|
| Age (years ± SD) | 39 ± 15 |
| Sex n (%) | |
| Male | 31 (60.8) |
| Female | 20 (39.2) |
| Ethnicity n (%) | |
| Non-Hispanic White | 36 (70.6) |
| Hispanic | 10 (19.6) |
| Non-Hispanic Black | 5 (9.8) |
| Allograft Laterality n (%) | |
| Left Kidney | 27 (56.3) |
| Right Kidney | 21 (43.7) |
| KDPI (% ± SD) | 45.5 ± 27 |
| Type of Donation n (%) | |
| Donation after Brain Death | 37 (75.5) |
| Donor after Circulatory Death | 12 (24.5) |
| Mean Ischemia Time (min ± SD) | 640 ± 309 |
| Mean HMP Time (min ± SD) | 477 ± 256 |
| Mean Cold Ischemia Time (min ± SD) | 1127 ± 405 |

SD: standard deviation; BMI: body mass index; KDPI: kidney donor profile index; HMP: hypothermic machine perfusion.

TABLE 2
sDNA levels and ex vivo organ timepoints.
sDNA Concentration (ng/ml) vs Ischemic Parameter

| Timepoint | Variable | Spearman Correlation (ρ) | p-value |
|---|---|---|---|
| 5 min | Static Ice Time | 0.0287 | 0.847 |
| | HMPT | 0.149 | 0.313 |
| | CIT | 0.1486 | 0.313 |
| Handoff | Static Ice Time | 0.0877 | 0.5404 |
| | HMPT | 0.238 | 0.0928 |
| | CIT | 0.3049 | 0.0296 |

HMP4T: hypothermic machine perfusion time; CIT: cold ischemia time (CIT).

### Association Between Ex Vivo Perfusion Variables and Post-Operative Graft Function

In keeping with previous findings that standard means of measuring HMP donor organ integrity are poor predictors of post-transplant clinical measure of graft function, Table 3 presents the association of these variables with early postoperative Cr, eGFR, and CRR. There was no significant relationship between RF and any clinical measure of early post-transplant graft function. In contrast, a significant correlation was observed between endpoint RR and Cr on all postoperative days up to day 4. Furthermore, neither RF or RR correlated significantly with CRR on any of the assessed postoperative days.

TABLE 3
Comparing post-transplant outcomes with renal flow,
renal resistance, and kidney donor profile index.

| | | | |
|---|---|---|---|
| Timepoint | Outcome Variable | Spearman Correlation (ρ) | p-value |

Endpoint Renal Flow

| | | | |
|---|---|---|---|
| POD 1 | Cr | −0.166 | 0.234 |
| | [symbol] GFR | 0.212 | 0.128 |
| | CRR | −0.157 | 0.263 |
| POD 2 | Cr | −0.0 [symbol] 7 | 0.486 |
| | [symbol] GFR | 0.091 | 0. [symbol] |
| | CRR | −0.119 | 0.39 [symbol] |
| POD 3 | Cr | −0.020 | 0. [symbol] |
| | [symbol] GFR | 0.007 | 0. [symbol] 59 |
| | CRR | −0.181 | 0.199 |
| POD 4 | Cr | −0.1 [symbol] 7 | 0. [symbol] |
| | [symbol] GFR | 0.210 | 0.183 |
| | CRR | −0.106 | 0. [symbol] 11 |

Endpoint Renal Resi [symbol] nce

| | | | |
|---|---|---|---|
| POD 1 | Cr | 0.309 | 0.027 |
| | [symbol] GFR | −0.256 | 0.069 |
| | CRR | 0.0 [symbol] 1 | 0.525 |
| POD 2 | Cr | 0. [symbol] | 0.031 |
| | [symbol] GFR | −0. [symbol] | 0.037 |
| | CRR | 0.0 [symbol] | 0. [symbol] |
| POD 3 | Cr | 0.216 | 0.140 |
| | [symbol] GFR | −0.23 [symbol] | 0. [symbol] 9 |
| | CRR | 0.112 | 0.441 |
| POD 4 | Cr | 0.122 | 0. [symbol] 55 |
| | [symbol] GFR | −0. [symbol] | 0.2 [symbol] |
| | CRR | 0.229 | 0.161 |

KDPI (%)

| | | | |
|---|---|---|---|
| POD 1 | Cr | 0.307 | 0. [symbol] |
| | [symbol] GFR | −0. [symbol] | 0.0 [symbol] |
| | CRR | −0. [symbol] | 0. [symbol] |
| POD 2 | Cr | 0.379 | 0.0061 |
| | [symbol] GFR | −0.394 | 0.0042 |
| | CRR | −0. [symbol] | 0.164 |
| POD 3 | Cr | 0.377 | 0.00 [symbol] |
| | [symbol] GFR | −0. [symbol] 92 | 0.00 [symbol] |
| | CRR | −0.161 | 0.26 [symbol] |
| POD 4 | Cr | 0.418 | 0.00 [symbol] 2 |
| | [symbol] GFR | −0.39 [symbol] | 0.012 |
| | CRR | −0.14 [symbol] | 0. [symbol] 77 |

[symbol]

indicates data missing or illegible when filed

HMP: hypothermic machine perfusion: POD: postoperative day: KDPI: kidney donor profile index; Cr: Creatinine; eGFR: estimated glomerular filtration rate: CRR: creatinine reduction ratio.

TABLE 4
Handoff sDNA concentration correlation with early graft function.

| Timepoint | Outcome Variable | Spearman Correlation (ρ) | p-value |
|---|---|---|---|
| POD 1 | Cr | 0.041 | 0.774 |
| | eGFR | −0.107 | 0.457 |
| | CRR | −0.390 | 0.0043 |
| POD 2 | Cr | 0.214 | 0.131 |
| | eGFR | −0.256 | 0.069 |
| | CRR | −0.351 | 0.0107 |
| POD 3 | Cr | 0.382 | 0.006 |
| | eGFR | −0.367 | 0.0088 |
| | CRR | −0.485 | 0.0003 |
| POD 4 | Cr | 0.330 | 0.043 |
| | eGFR | −0.373 | 0.019 |
| | CRR | −0.500 | 0.0012 |

POD: postoperative day; Cr: creatinine; eGFR: estimated glomerular filtration rate; CRR: creatinine reduction ratio.

### Association Between KDPI and Post-Operative Graft Function

The Kidney Donor Profile Index (KDPI) is a cumulative percentile measure that characterizes the donor associated risk of post-transplant graft failure and aids transplant physicians in their decision to transplant a graft.²⁰ However, the impact of HMP on KDPI's association with early graft function is still unknown. When the KDPI was examined, statistically significant correlations were observed between KDPI and Cr, as well as eGFR on all postoperative days (Table 3). In contrast, there was no notable statistically significant relationship between KDPI and CRR as a measure of early post-operative graft function.

Perfusion Parameters Correlate with Perfusate sDNA

Next, it was investigated whether standard perfusion measures that are currently used to assess organ quality correlated with perfusate sDNA levels. To do so, a comparison was made of sDNA at five minutes and at handoff to RR and RF at the start of HMP, at 2-hour and 4-hour time points, and at the conclusion of HMP (Table 5). Five-minute (5 min) perfusate sDNA levels correlated positively with graft RF on the pump at 2 hours and 4 hours, while handoff sDNA correlated with 2-hour, 4-hour, and final RF (Table 5, FIG. 5). There was a negative trend in the correlations between RR and sDNA at 5 min and at handoff, although these were not significant.

TABLE 5
Perfusate sDNA vs. hypothermic machine perfusion parameters
sDNA Concentration and HMP Parameters

| | | Spearman | |
|---|---|---|---|
| Timepoint | Variable | Correlation (p) | p-value |
| 5 min | Initial RR | −0.145 | 0.320 |
| | 2 hrRR | −0.272 | 0.074 |
| | 4 hrRR | −0.135 | 0.413 |
| | Final RR | −0.115 | 0.441 |
| Handoff | Initial RR | 0.0063 | 0.965 |
| | 2 hrRR | −0.200 | 0.178 |
| | 4 hrRR | −0.109 | 0.498 |
| | Final RR | −0.186 | 0.199 |
| 5 min | Initial RF | 0.215 | 0.143 |
| | 2 hrRF | 0.334 | 0.023 |
| | 4 hrRF | 0.311 | 0.051 |
| | Final RF | 0.277 | 0.057 |
| Handoff | Initial RF | 0.128 | 0.371 |
| | 2 hrRF | 0.391 | 0.0055 |
| | 4 hrRF | 0.365 | 0.017 |
| | Final RF | 0.364 | 0.0087 |

HMP: Hypothermic machine perfusion; RR: renal vascular resistance; RF: renal vascular flow; 5 min: concentration of sDNA at 5 minutes hypothermic machine perfusion; Handoff: concentration of sDNA at endpoint hypothermic machine perfusion.

Perfusate sDNA Levels Correlate with Measures of Early Graft Function

It was then assessed whether perfusate sDNA levels correlate with post-transplant function. There was a statistically significant correlation between 5 min sDNA and post-transplant graft function, with higher 5 min perfusate sDNA concentrations correlating with lower CRR on POD2 and POD4 (Table 6, FIG. 6).

TABLE 6
Five-minute sDNA concentration compared with early graft function
5 min sDNA Concentration and Early Graft Function

| | Variable vs | | |
|---|---|---|---|
| | 5 min sDNA | Spearman | |
| Timepoint | (ng/ml) | Correlation (ρ) | P-value |
| POD 1 | Cr | −0.049 | 0.743 |
| | eGFR | −0.066 | 0.646 |
| | CRR | −0.254 | 0.072 |
| POD 2 | Cr | 0.121 | 0.413 |
| | eGFR | −0.127 | 0.389 |
| | CRR | −0.314 | 0.025 |
| POD 3 | Cr | 0.123 | 0.409 |
| | eGFR | −0.114 | 0.441 |
| | CRR | −0.267 | 0.061 |
| POD 4 | Cr | 0.244 | 0.098 |
| | eGFR | −0.144 | 0.396 |
| | CRR | −0.342 | 0.033 |

Importantly, grafts with higher concentration of perfusate sDNA at time of graft handoff had significantly reduced early post-transplant graft function (Table 4, FIG. 2). Specifically, higher handoff sDNA concentrations correlated strongly with lower CRR on all postoperative days, lower POD3 eGFR, and higher POD3 and POD4 serum Cr levels.

sDNA Levels Predict Delayed Graft Function

Finally, the relationship between sDNA in HMP perfusate and the development of DGF postoperatively was examined. There was a significantly higher level of handoff sDNA (p=0.018) and 5 min sDNA (p=0.047) in the kidneys whose recipients ultimately exhibited DGF in comparison with the kidneys that did not exhibit DGF (FIG. 3A, 3B). The receiver operating characteristics (ROC) curve for our cohort determined that sDNA concentration as a biomarker for DGF has a reliable prognostic performance with an area under the curve (AUC) of 0.816 (95% CI 0.68-0.96; p=0.021) in samples obtained at kidney handoff to surgeon, and an AUC of 0.771 (95% CI 0.58-0.96; p=0.048) in samples obtained at 5 min of perfusion. Based on a Youden index/ROC curve analysis of the cohort, using 2.69 ng/ml of sDNA in the handoff perfusate as the threshold for the likelihood of post-transplant DGF yields 100% sensitivity (95% CI 0.57-1.00) and 64.7% specificity (95% CI 0.51-0.76) (FIG. 3C, 3D). In comparison, the ROC curves generated for KDPI, RR, and RF in predicting DGF were less reliable and were not statistically significant with this cohort (FIG. 3E, 3F, 3G).

### REFERENCES

1. Moers C, Smits J M, Maathuis M H, Treckmann J, van Gelder F, Napieralski B P, et al. Machine perfusion or cold storage in deceased-donor kidney transplantation. N Engl J Med. 2009 Jan. 1; 360(1):7-19. doi: 10.1056/NEJMoa0802289. PMID: 19118301.
2. Tingle S J, Figueiredo R S, Moir J A, Goodfellow M, Talbot D, Wilson C H. Machine perfusion preservation versus static cold storage for deceased donor kidney transplantation. Cochrane Database Syst Rev. 2019 Mar. 15; 3(3):CD011671. doi: 10.1002/14651858.CD011671.pub2. PMID: 30875082; PMCID: PMC6419919.
3. Korayem I M, Agopian V G, Lunsford K E, Gritsch H A, Veale J L, Lipshutz G S, et al., Factors predicting kidney delayed graft function among recipients of simultaneous liver-kidney transplantation: A single-center experience. Clin Transplant. 2019 June; 33(6):e13569. doi: 10.1111/ctr.13569. Epub 2019 May 7. PMID: 31006141; PMCID: PMC6653637.
4. Kruszyna T, Richter P. Hypothermic Machine Perfusion of Kidneys Compensates for Extended Storage Time: A Single Intervention With a Significant Impact. Transplant Proc. 2021 April; 53(3):1085-1090. doi: 10.1016/j.transproceed.2021.01.022. Epub 2021 Feb. 10. PMID: 33579549.
5. Lam N N, Boyne D J, Quinn R R, Austin P C, Hemmelgarn B R, Campbell P, et al. Mortality and Morbidity in Kidney Transplant Recipients With a Failing Graft: A Matched Cohort Study. Can J Kidney Health Dis. 2020 Apr. 14; 7:2054358120908677. doi: 10.1177/2054358120908677. PMID: 32313663; PMCID: PMC7158256.
6. Henry M L, Sommer B G, Ferguson R M. Renal blood flow and intrarenal resistance predict immediate renal allograft function. Transplant Proc 1986; 18: 557.
7. Sonnenday C J, Cooper M, Kraus E, Gage F, Handley C, Montgomery R A. The hazards of basing acceptance of cadaveric renal allografts on pulsatile perfusion parameters alone. Transplantation. 2003 Jun. 27; 75(12):2029-33. doi: 10.1097/01.TP.0000065296.35395.FD. PMID: 12829906.
8. Mozes M F, Skolek R B, Korf B C. Use of perfusion parameters in predicting outcomes of machine-preserved kidneys. Transplant Proc. 2005 January-February; 37(1):350-1. doi: 10.1016/j.transproceed.2005.01.058. PMID: 15808640.
9. Singh N, Logan A, Schenk A, Bumgardner G, Brock G, El-Hinnawi A, et al. Machine perfusion of kidney allografts affects early but not late graft function. Am J Surg. 2022 April; 223(4):804-811. doi: 10.1016/j.amjsurg.2021.06.019. Epub 2021 Jul. 6. PMID: 34253338; PMCID: PMC9017432.
10. Hosgood S A, Barlow A D, Hunter J P, Nicholson M L. Ex vivo normothermic perfusion for quality assessment of marginal donor kidney transplants. Br J Surg. 2015 October; 102(11):1433-40. doi: 10.1002/bjs.9894. Epub 2015 Aug. 27. PMID: 26313559.
11. Bellini M I, Tortorici F, Amabile M I, D'Andrea V. Assessing Kidney Graft Viability and Its Cells Metabolism during Machine Perfusion. Int J Mol Sci. 2021 Jan. 23; 22(3):1121. doi: 10.3390/ijms22031121. PMID: 33498732; PMCID: PMC7865666.
12. De Beule J, Jochmans I. Kidney Perfusion as an Organ Quality Assessment Tool—Are We Counting Our Chickens Before They Have Hatched?J Clin Med. 2020 Mar. 23; 9(3):879. doi: 10.3390/jcm9030879. PMID: 32210197; PMCID: PMC7141526.
13. van Smaalen T C, Hoogland E R, van Heurn L W. Machine perfusion viability testing. Curr Opin Organ Transplant. 2013 April; 18(2):168-73. doi: 10.1097/MOT.0b013e32835e2a1b. PMID: 23385886.
14. Guzzi F, Knight S R, Ploeg R J, Hunter J P. A systematic review to identify whether perfusate biomarkers produced during hypothermic machine perfusion can predict graft outcomes in kidney transplantation. Transpl Int. 2020 June; 33(6):590-602. doi: 10.1111/tri.13593. Epub 2020 Feb. 28. PMID: 32031281.
15. Parikh C R, Hall I E, Bhangoo R S, Ficek J, Abt P L, Thiessen-Philbrook H, et al.

Associations of Perfusate Biomarkers and Pump Parameters With Delayed Graft Function and Deceased Donor Kidney Allograft Function. Am J Transplant. 2016 May; 16(5):1526-39. doi: 10.1111/ajt.13655. Epub 2016 Feb. 17. PMID: 26695524; PMCID: PMC4844819.
16. Kanou T, Nakahira K, Choi A M, Yeung J C, Cypel M, Liu M, et al. Cell-free DNA in human cx vivo lung perfusate as a potential biomarker to predict the risk of primary graft dysfunction in lung transplantation. J Thorac Cardiovasc Surg. 2021 August; 162(2):490-499.e2. doi: 10.1016/j.jtcvs.2020.08.008. Epub 2020 Aug. 11. PMID: 32928548.
17. Boominathan V, Willman M, Battula N, Zarrinpar A, Duarte S. Perfusate Cell-Free DNA Content is a Potential Marker of Cellular Injury During Hypothermic Machine Perfusion of Porcine Kidneys Subject to Prolonged Warm Ischemia [abstract]. Am J Transplant. 2022; 22 (suppl 3). https://atcmeetingabstracts.com/abstract/perfusate-cell-free-dna-content-is-a-potential-marker-of-cellular-injury-during-hypothermic-machine-perfusion-of-porcine-kidneys-subject-to-prolonged-warm-ischemia/. Accessed Nov. 17, 2022.
18. Sedigh A, Tufveson G, Backman L, Biglarnia A R, Lorant T. Initial experience with hypothermic machine perfusion of kidneys from deceased donors in the Uppsala region in Sweden. Transplant Proc. 2013 April; 45(3):1168-71. doi: 10.1016/j.transproceed.2012.10.017. PMID: 23622652.
19. Patel S K, Pankewycz O G, Nader N D, Zachariah M, Kohli R, Laftavi M R. Prognostic utility of hypothermic machine perfusion in deceased donor renal transplantation. Transplant Proc. 2012 September; 44(7):2207-12. doi: 10.1016/j.transproceed.2012.07.129. PMID: 22974956.
20. Howard D H. Why do transplant surgeons turn down organs?A model of the accept/reject decision. J Health Econ. 2002 November; 21(6):957-69. doi: 10.1016/s0167-6296(02)00077-2. PMID: 12475120.
21. Butt A N, Swaminathan R. Overview of circulating nucleic acids in plasma/serum. Ann N Y Acad Sci. 2008 August; 1137:236-42. doi: 10.1196/annals.1448.002. PMID: 18837954.
22. Swarup V, Rajeswari M R. Circulating (cell-free) nucleic acids—a promising, non-invasive tool for early detection of several human diseases. FEBS Lett. 2007 Mar. 6; 581(5):795-9. doi: 10.1016/j.febslet.2007.01.051. Epub 2007 Feb. 2. PMID: 17289032.
23. Ranucci R. Cell-Free DNA: Applications in Different Diseases. Methods Mol Biol. 2019; 1909:3-12. doi: 10.1007/978-1-4939-8973-7_1. PMID: 30580419.
24. Gielis E M, Ledeganck K J, De Winter B Y, Del Favero J, Bosmans J L, Claas F H, et al. Cell-Free DNA: An Upcoming Biomarker in Transplantation. Am J Transplant. 2015 October; 15(10):2541-51. doi: 10.1111/ajt.13387. Epub 2015 Jul. 16. PMID: 26184824.
25. Beck J, Oellerich M, Schulz U, Schauerte V, Reinhard L, Fuchs U, et al. Donor-Derived Cell-Free DNA Is a Novel Universal Biomarker for Allograft Rejection in Solid Organ Transplantation. Transplant Proc. 2015 October; 47(8):2400-3. doi: 10.1016/j.transproceed.2015.08.035. PMID: 26518940.
26. Oellerich M, Shipkova M, Asendorf T, Walson P D, Schauerte V, Mettenmeyer N, et al. Absolute quantification of donor-derived cell-free DNA as a marker of rejection and graft injury in kidney transplantation: Results from a prospective observational study. Am J Transplant. 2019 November; 19(11):3087-3099. doi: 10.1111/ajt.15416. Epub 2019 May 28. PMID: 31062511; PMCID: PMC6899936.
27. Swanson K J, Aziz F, Garg N, Mohamed M, Mandelbrot D, Djamali A, et al. Role of novel biomarkers in kidney transplantation. World J Transplant. 2020 Sep. 18; 10(9):230-255. doi: 10.5500/wjt.vl0.i9.230. PMID: 32995319; PMCID: PMC7504189.
28. Govani M V, Kwon 0, Batiuk T D, Milgrom M L, Filo R S. Creatinine reduction ratio and 24-hour creatinine excretion on posttransplant day two: simple and objective tools to define graft function. J Am Soc Nephrol. 2002 June; 13(6):1645-9. doi: 10.1097/01.asn.0000014253.40506.f6. PMID: 12039994.
29. Rodrigo E, Ruiz J C, Pinera C, Femndez-Fresnedo G, Escallada R, Palomar R, et al. Creatinine reduction ratio on post-transplant day two as criterion in defining delayed graft function. Am J Transplant. 2004 July; 4(7):1163-9. doi: 10.1111/j.1600-6143.2004.00488.x. PMID: 15196076.
30. Vilar E, Varagunam M, Yagoob M M, Raftery M, Thuraisingham R. Creatinine reduction ratio: a useful marker to identify medium and high-risk renal transplants. Transplantation. 2010 Jan. 15; 89(1):97-103. doi: 10.1097/TP.0b013e3181be3dd1. PMID: 20061925.

What is claimed is:

1. A system for ex vivo organ perfusion comprising:
i. an organ chamber for receiving and supporting an organ and a perfusate,
ii. a perfusate pump positioned and arranged to regulate supply of the perfusate to the organ and to collect perfusate from the organ,
iii. a pressure controller connected to said perfusate pump for maintaining a pressure level in the perfusate within a predetermined range,
iv. an oxygenator connected to said perfusate pump for maintaining an oxygen level in the perfusate supplied to the organ by said perfusate pump within a predetermined range,
v. a temperature regulator connected to said perfusate pump and positioned and arranged to maintain temperatures of the organ and the perfusate within predetermined ranges, and
vi. a cfDNA detector connected to said perfusate pump and positioned and arrange to quantify cfDNA levels in the perfusate.

2. The system of claim 1, wherein the organ comprises an allograft, an autograft, an isograft, or a xenograft.

3. The system of claim 2, wherein the organ is a kidney.

4. The system of any of claims 1-3, wherein the cfDNA detector quantifies the cfDNA by digital PCR, qPCR, UV spectrophotometry, or fluorometry.

5. The system of any of claims 1-4, wherein the system further comprises a filter positioned in the system to filter perfusate before circulating through the cfDNA detector.

6. The system of any of claims 1-5, further comprising an infusion port connected to the perfusate pump.

7. The system of any of claim 1-6, further comprising a sample collection port connected to the perfusate pump.

8. The system of any of any of claims 1-7, wherein the perfusate comprises UW solution, Bretschneider solution, BES-HMP solution, perfluorocarbon solutions, STEEN solution (albumin and Dextran 40 based), blood or BGP-35 solution.

9. The system of any of claims 1-8, wherein the pressure controller maintains a pressure of 30 to 100 mmHg²⁰.

10. The system of system of any of claims 1-9, wherein the temperature regulator maintains a temperature of about 0° C. to 12° C.

11. A device for quantifying cfDNA in an ex vivo organ perfusate.

12. The device of claim 11, wherein the device can connect to a hypothermic perfusion machine.

13. The device of claims 11 or 12, wherein the device quantifies cfDNA by digital PCR, qPCR, UV spectrophotometry, or fluorometry.

14. The device of any of claims 11-13, wherein the perfusate is filtered before quantification.

15. A method for monitoring pre-transplant organ function comprising connecting an organ to a hypothermic perfusion machine, circulating a perfusate through the organ, filtering the perfusate, and measuring cfDNA in the perfusate, wherein a level of cfDNA increased at least 1%, 5%, 10%, 20%, 30% or higher relative to control levels or higher than a predetermined threshold level indicates poor pre-transplant organ function.

16. The method of claim 15, wherein the organ comprises an allograft, an autograft, an isograft, or a xenograft.

17. The method of claims 15 or 16, wherein the organ is a kidney.

18. The method of any of claims 15-17, wherein cfDNA is measured by digital PCR, qPCR, UV spectrophotometry, or fluorometry.

19. The method of any of claims 15-18, wherein the perfusate comprises UW solution, Bretschneider solution, BES-HMP solution, or BGP-35 solution.

20. The method of any of claims 15-19, wherein the predetermined threshold level is about 2.0 to about 3.0 ng/ml in the perfusate.

21. The method of any of claims 15-20, further comprising implanting the organ into a patient if the organ is determined to not have poor pre-transplant organ function, or implanting the organ into the patient and, if the organ is a kidney, administering dialysis within 1-48 hours of implantation if the organ is determined to have poor pre-transplant organ function.
